(19) United States
(12) Patent Application Publication
(10) Pub. No.: US 2020/0368277 A1
(43) Pub. Date: Nov. 26, 2020

(54) T-CELL RECEPTOR SEQUENCES FOR ACTIVE IMMUNOTHERAPY

(71) Applicant: POLYBIOCEPT GMBH, Alzenau (DE)

(72) Inventors: Markus MAEURER, Äkersberga (SE); Ernest DODOO, Stockholm (SE)

(73) Assignee: POLYBIOCEPT GMBH, Alzenau (DE)

(21) Appl. No.: 16/076,063

(22) PCT Filed: Feb. 8, 2017
(86) PCT No.: PCT/EP2017/052785

§ 371 Date: Aug. 7, 2018

(30) Foreign Application Priority Data
Feb. 8, 2016 (EP) 16154713.8

(51) Int. Cl. A61K 35/17; C07K 14/725; C12N 5/0783

(52) U.S. Cl. CPC A61K 35/17; A61K 38/00; C12N 5/0636; C07K 14/7051

(57) ABSTRACT

The present invention provides a protein, in particular a TCR comprising a Vβ amino acid sequence with a sequence identity of at 90%, to a sequence selected from SEQ ID NO: 1 to 27, a T-cell comprising the TCR a method of selecting a T-cell product for use in active immunotherapy based on the identification of an Vβ amino acid sequence with a sequence identity of at 90%, to a sequence selected from SEQ ID NO: 1 to 27 in a T-cell product.

### FIELD OF THE INVENTION

The present invention relates to proteins comprising a Vbeta sequence with an amino acid sequence with a sequence identity of at 90%, to a sequence for use in immunotherapy.

### BACKGROUND OF THE INVENTION

One of the most promising advances for the treatment of cancer is a new therapeutic class called active cellular immunotherapy (ACI). Active immunotherapies stimulate the patient's immune system with the intent of promoting an antigen specific anti-tumor effect using the body's own immune cells. In addition active immunotherapies seek to create durable anti-tumor response that can protect against minimal residual disease and tumor recurrences as well as against potentially pre-malignant tumor lesions.

Clinically relevant and long-term remissions have been achieved in patients with melanoma using T-cells directed against tumors (tumor reactive T-cells) (1, 2). These approaches usually rely on the harvesting of tumor infiltrating lymphocytes (TIL) from tumor lesions or T-cells from peripheral blood.

Clinical (antitumor) efficacy appears to be mediated by CD8+, or by CD4+ T-cells residing preferentially in central memory cells, defined by CD45RA-CCR7+, defined ex vivo from patients responding to T-cell based therapy. The phenotype of such T-cells is determined by the ex vivo expanded T-cell population, as well as by host factors after adoptive transfer. A diverse population of T-cells targeting cancer cells may be advantageous for effective immune responses, including long-term memory T-cells, yet also T-cells that can immediately react to (cancer) target cells and produce anti-tumor directed immune responses, including terminally differentiated T-cells that express cytolytic molecules, such as granzyme and perforin (3, 4). Long term-memory immune memory is in part determined by the increased proliferation potential and half-life that can be measured by the telomere length (5, 6).

In WO 2015/189357 A1 the inventors have described a novel cytokine cocktail containing IL-2, IL-15 and IL-21 for the expansion of lymphocytes, in particular T-cells. T-cell populations obtained by expansion in the presence of the cytokine cocktail are able to not only recognize autologous tumor calls but also kill such tumor cells in vitro.

WO 2015/189357 A1 describes a variety of T-cell products obtained from an expansion of T-cells from the tumor, i.e. tumor infiltrating lymphocytes (TILs) or peripheral blood of patients with pancreatic cancer or glioblastoma. With the expansion protocol using the cytokine cocktail containing IL-2, IL-15 and IL-21 it is possible to produce several T-cell products in parallel. These T-cell products in general show a phenotype distribution advantageous for the active immune therapy.

However, besides the phenotype distribution and the in vitro testing of the T-cell product the clinician at present does not have further determinants for the selection for a T-cell product for insertion into the patient.

### SUMMARY OF THE INVENTION

The present invention is inter alia based on the identification TCR sequences that are responsible for recognition and killing of diseased tissue cells. TCRs with the respective TCR sequences target tumor associated antigens and T-cells comprising the TCRs are therefore valuable therapeutic tools and a detection of the TCR sequences can be used for evaluation of T-cell products, i.e. an in vitro expanded T-cell population for therapeutic use.

Thus, according to a first aspect the invention provides a protein comprising a Vβ amino acid sequence with a sequence identity of at 90% to a sequence selected from SEQ ID NO: 1 to 27.

According to a second aspect the invention provides a T-cell comprising a TCR which is a protein according to the first aspect. T-cell according to the second aspect may in particular be used in the treatment of cancer.

Furthermore according to a third aspect the invention provides a method of selecting a T-cell product for use in active immunotherapy in a patient, wherein the method comprises the following steps:
a) providing one or more T-cell products;
b) identifying the presence of a TCR comprising a sequence with a homology of at least 95% to the sequence selected from SEQ ID NO: 1 to 27 in the one or more T-cell products; and
c) selecting the T-cell product comprising a TCR identified in step b) for use in therapy.

### DESCRIPTION OF THE DRAWINGS

FIG. 1 shows the frequency of IFNγ and TNFα producing TIL in response to autologous tumor cells. Numbers represent the percentage of T-cells specifically producing IFNγ (black circles) and TNFα (open circles) in the parental CD8+, CD4+, or (CD3+), CD4-CD8− TIL subsets. Each circle represent TIL from an individual patient. Medium values have been subtracted. Right panel: example flow cytometric analysis of CD4+ and CD8+ TIL producing cytokines directed against autologous tumor cells.

FIG. 2 shows the link of expanded Vβ families in TIL with the recognition of autologous tumor cells, in particular the frequency of TNFalpha producing T-cells in CD3+CD4+ T-cells. Top left: positive control. Middle panel. Exposure to autologous tumor cells. TIL gated on CD3+CD4+ T-cells: 3.5% cells produce TNFalpha; right: 13.6% TNFα producing TIL in DNCD3+CD4+ Vβ2+ T-cells from patient GBM-D. Bottom panel. Left: negative control in CD3+CD4+ TIL. Right: CD3+CD4+V62 2+ TIL.

FIG. 3 shows (A) the IFNγ production in TILs after co-culture with autologous tumor cells (Panc 9). CD8+ TILs (Vβ13.2 dominant) recognize the autologous tumor and IFNγ production can be inhibited by the anti-MHC-I antibody (W6/32), yet no with the anti-HLA-DR directed mAb. (B) The Recognition of the autologous tumor cell line in Panc 17 TIL using a standard Chromium 51 release assay.

FIG. 4 shows the results of a TCR CDR3 analysis of frequent TCR Vβ families after a 4 week expansion using IL-2, IL-15 and IL-21.

### DETAILED DESCRIPTION OF THE INVENTION

As commonly used in the art and used herein, a “T-cell receptor”, or “TCR”, is a molecule found on the surface of T cells, or T lymphocytes, that is responsible for recognizing fragments of antigen as peptides bound to major histocompatibility complex (MHC) molecules. The TCR is a heterodimer. There are two types the αβ TCR consisting of an alpha (α) chain and a beta (β) chain (in humans in 95%) and a γδTCR consisting of a gamma ( ) and delta ( ) chain (in humans 5%). When the TCR engages with antigenic peptide and MHC (peptide/MHC), the T lymphocyte is activated through signal transduction, mediated by associated enzymes, co-receptors, specialized adaptor molecules, and activated or released transcription factors. The variable TCR receptor is part of octomeric complex including additionally the three dimeric signaling modules CD3δ/ε, CD3γ/ε and CD247 ζ/ζ or ζ/η.

The TCR α and β chains are both composed of a variable part and a constant part. The diversity is based mainly on genetic recombination of the DNA encoded segments in individual somatic T cells—either somatic V(D)J recombination using RAG1 and RAG2 recombinases or gene conversion using cytidine deaminases (AID). V(D)J recombination occurs in the primary lymphoid organs (bone marrow for B cells and thymus for T cells) and in a nearly random fashion rearranges variable (V), joining (J), and in some cases, diversity (D) gene segments. The process ultimately results in novel amino acid sequences in the antigen-binding regions of Igs and TCRs that allow for the recognition of antigens.

The term “Vbeta” or “Vβ” as used herein relates to the variable part of the beta chain of the TCR composed of the gene products of the variable (V), joining (J), and in some cases, diversity (D) gene segments.

As will be understood by the skilled person, the “T-cell receptor”, and “TCR” relate both to naturally occurring receptors of T-cell receptors and recombinantly expressed T-cell receptors.

In agreement with the general understanding in the art will T cell, or T lymphocyte, is a type of lymphocyte (a subtype of white blood cell) that plays a central role in cell-mediated immunity. T cells can be distinguished from other lymphocytes, such as B cells and natural killer cells, by the presence of a T-cell receptor on the cell surface. They are called T cells because they mature in the thymus from thymocytes.

“Disease associated antigens” according to the invention are antigens involved in a disease. Accordingly, disease associated antigens can be tumor-associated antigens TAA.

According to the invention an “antigen” (Ag) is any structural substance which serves as a target for the receptors of an adaptive immune response, TCR or antibody, respectively. Antigens are in particular proteins, polysaccharides, lipids and substructures thereof such as peptides. Lipids and nucleic acids are in particular antigenic when combined with proteins or polysaccharides.

“Tumor associated antigens” or “TAA” according to the invention are antigens that are presented by MHC I or MHC II molecules or non-classical MHC molecules on the surface of tumor cells. As used herein TAA includes “tumor-specific antigens” which are found only on the surface of tumor cells, but not on the surface of normal cells.

As used herein, “interleukin 2” or “IL-2” refers to human IL-2 as defined by accession number P60568 of the UniProtKB database (http://www.uniprot.org/uniprot/) and functional equivalents thereof. Functional equivalents of IL-2 include relevant substructures or fusion proteins of IL-2 that remain the functions of IL-2. Accordingly, the definition IL-2 comprises any protein with a sequence identity to UniProtKB P60568-1of at least 80%, preferably at least 90%, more preferably at least 95%, most preferably at least 98%. Recombinant human IL-2 produced in E. coli as a single, non-glycosylated polypeptide chain with 134 amino acids and having a molecular mass of 15 kDa is commercially available in lyophilized form from Prospec as CYT-209.

As used herein, “interleukin 15” or “IL-15” refer to human IL-15 as defined by accession number P40933 of the UniProtKB database and functional equivalents thereof. Functional equivalents of IL-15 include relevant substructures or fusion proteins of IL-15 that remain the functions of IL-15. Accordingly the definition IL-15 comprises any protein with a sequence identity to UniProtKB P40933-1 of at least 80%, preferably at least 90%, more preferably at least 95%, most preferably at least 98%. Recombinant human IL-15 produced in E. coli as a single, non-glycosylated polypeptide chain with 114 amino acids (and an N-terminal Methionine) and having a molecular mass of 12.8 kDa is commercially available in lyophilized form from Prospec as CYT-230.

As used herein, “interleukin 21” or “IL-21” refer to human IL-21 as defined by accession number Q9HBE4 of the UniProtKB database and functional equivalents thereof. Functional equivalents of IL-21 include relevant substructures or fusion proteins of IL-21 that remain the functions of IL-21. Accordingly the definition IL-21 comprises any protein with a sequence identity to UniProtKB Q9HBE4-1 of at least 80%, preferably at least 90%, more preferably at least 95%, most preferably at least 98%. Recombinant human IL-21 produced in E. coli as a single, non-glycosylated polypeptide chain with 132 amino acids and having a molecular mass of 15 kDa is commercially available in lyophilized form from Prospec as CYT-408.

A “peptide” as used herein may be composed of any number of amino acids of any type, preferably naturally occurring amino acids, which, preferably, are linked by peptide bonds. In particular, a peptide comprises at least 3 amino acids, preferably at least 5, at least 7, at least 9, at least 12, or at least 15 amino acids. Furthermore, there is no upper limit for the length of a peptide. However, preferably, a peptide according to the invention does not exceed a length of 500 amino acids, more preferably it does not exceed a length of 300 amino acids; even more preferably it is not longer than 250 amino acids.

Thus, the term “peptide” includes “oligopeptides”, which usually refer to peptides with a length of 2 to 10 amino acids, and “polypeptides” which usually refer to peptides with a length of more than 10 amino acids.

The term “protein” refers to a peptide with at least 60, at least 80, preferably at least 100 amino acids.

The term “fusion protein” according to the invention relates to proteins created through the joining of two or more genes, cDNAs or sequences that originally coded for separate proteins/peptides. The genes may be naturally occurring in the same organism or different organisms or may be synthetic polynucleotides.

The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using thenobrief option) is used as the percent identity and is calculated as follows:

(Identical Residues×100)/(Length of Alignment−Total Number of Gaps in Alignment)

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim, except for impurities ordinarily associated therewith. When the phrase “consists of” appears in a clause of the body of a claim, rather than immediately following the preamble, it limits only the element set forth in that clause; other elements are not excluded from the claim as a whole. The transitional phrase “consisting essentially of” limits the scope of a claim to the specified materials or steps “and those that do not materially affect the basic and novel characteristic(s)” of the claimed invention. “A ‘consisting essentially of’ claim occupies a middle ground between closed claims that are written in a ‘consisting of’ format and fully open claims that are drafted in a ‘comprising’ format.”

“Expansion” or “clonal expansion”, as used herein, means production of daughter cells all arising originally from a single cell. In a clonal expansion of lymphocytes, all progeny share the same antigen specificity.

A “T-cell product”, as used herein, refers to a population of T-cell for use in immune therapy.

“TIL” according to the invention refers to tumor infiltrating lymphocyte. These are lymphocytes, in particular T-cells predominantly found in a tumor. A lymphocyte sample derived from tumor is also referred as TIL. TIL also relates to any kind of lymphocyte that is located in, on or around a tumor.

“Clinical/biological relevance” as used herein relates to the ability of a T-cell to provide at least one of the following: containment of tumor cells, destruction of tumor cells, prevention of metastasis, stop of proliferation, stop of cellular activity, stop of progress of cells to malignant transformation, prevention of metastases and/or tumor relapse, including reprogramming of malignant cells to their non-malignant state; prevention and/or stop of negative clinical factors associated with cancer, such as malnourishment or immune suppression, stop of accumulation of mutations leading to immune escape and disease progression, including epigenetic changes, induction of long-term immune memory preventing spread of the disease or future malignant transformation affecting the target (potential tumor cells), including connective tissue and non-transformed cells that would favor tumor disease.

According to a first aspect, the invention provides a protein comprising a Vβ amino acid sequence with a sequence identity of at 90%, to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 26. The sequences SEQ ID NO: 1 to 26 were identified in T-cell products obtained by expansion of T-cells and shown to be responsible for recognition and killing of autologous tumor cells. The sequences are shown in Tables 1 and 2.

TABLE 1
Vβ sequences found in TIL
expanded from Glioblastoma

GBM A-CD4 (SEQ ID NO: 1)
GAVVSQHPSWVICKSGTSVKIECRSLDFQATTMFWYRQFPKQ
SLMLMATSNEGSKATYEQGVEKDKFLINHASLTLSTLTVTSA
HPEDSSFYICSAATGDRPYEQYFGPGTRLTVT
GBM A-CD8 A (SEQ ID NO: 2)
DAGITQSPRHKVTETGTPVTLRCHQTENHRYMYWYRQDPGH
GLRLIHYSYGVKDTDKGEVSDGYSVSRSKTEDFLLTLESATS
SQTSVYFCAIRTGSDNEQSFGPGTRLTVL
GBM A-CD8 B (SEQ ID NO: 3)
EAEVAQSPRYKITEKSQAVAFWCDPISGHATLYWYRQILGQ
GPELLVQFQDESVVDDSQLPKDRFSAERLKGVDSTLKIQPA
ELGDSAMYLCASRYTGSIEQFFGPGTRLTVL
GBM F-CD4 (SEQ ID NO: 4)
EAQVTQNPRYLITVTGKKLTVTCSQNMNHEYMSWYRQDPGL
GLRQIYYSMNVEVTDKGDVPEGYKVSRKEKRNFPLILESPSP
NQTSLYFCASSAGTSGVTYEQYFGPGTRLTVT
GBM G-CD4 A (SEQ ID NO: 5)
NAGVTQTPKFQVLKTGQSMTLQCAQDMNHNSMYWYRQDPGM
GLRLIYYSASEGTTDKGEVPNGYNVSRLNKREFSLRLESAAP
SQTSVYFCASSTRFEQYFGPGTRLTVT
GBM G-CD4 B (SEQ ID NO: 6)
NAGVTQTPKFQVLKTGQSMTLQCAQDMNHEYMSWYRQDPGM
GLRLIHYSVGAGITDQGEVPNGYNVSRSTTEDFPLRLLSAAP
SQTSVYFCASSTRFEQYFGPGTRLTVT
GBM G CD4 C (SEQ ID NO: 7)
NAGVTQTPKFRILKIGQSMTLQCTQDMNHNYMYWYRQDPGM
GLKLIYYSVGAGITDKGEVPNGYNVSRSTTEDFPLRLELAAP
SQTSVYFCASSTRFEQYFGPGTRLTVT
GBM H-CD8 A (SEQ ID NO: 8)
DAGVIQSPRHEVTEMGQEVTLRCKPISCHNSLFWYRQTMMR
GLELLIYFNNNVPIDDSGMPEDRFSAKMPNASFSTLKI
QPSEPRDSAVYFCASSSRRDHTYNGQFFGPGTRLTVL
GBM H-CD8 B (SEQ ID NO: 9)
DAGVIQSPRHEVTEMGQEVTLRCKPISGHDYLFWYRQTMMR
GLELLIYFNNNVPIDDSGMPEDRFSAKMPNASFSTLKIQPSE
PRDSAVYFCASSSRRDHTYNGQFGPGTRLTVL
GBM H-CD8 C (SEQ ID NO: 10)
EAQVTQNPRYLITVTGKKLTVTCSQNMNHEYMSWYRQDPGL
GLRQIYYSMNVEVTDKGDVPEGYKVSRKEKRNFPLILESPSP
NQTSLYFCASSLQGANYGYTFGSGTRLTVV
GBM I-CD4 (SEQ ID NO: 11)
GAVVSQHPSWVICKSGTSVKIECRSLDFQATTMFWYRQFPK
QSLMLMATSNEGSKATYEQGVEKDKFLINHASLTLSTLTVTS
AHPEDSSFYICSARVIPSGGVVQGTDTQYFGPGTRLTVL
GBM I-CD8 (SEQ ID NO: 12)
KAGVTQTPRYLIKTRGQQVTLSCSPISCHRSVSWYQQTPGQ
GLQFLFEYFSETQRNKGNFPGRFSGRQFSNSRSEMNVSTLEL
GDSALYLCASSLSWDKSYEQYFGPGTRLTVT
GBM J-CD4 (SEQ ID NO: 13)
EAEVAQSPRYKITEKSQAVAFWCDPISGHATLYWYRQILGQ
GPELLVQFQDESVVDDSQLPKDRFSAERLKGVDSTLKIQPA
ELGDSAMYLCASSLSRLALFSYEQYFGPGTRLTVT

TABLE 2
Vβ sequences found in TIL
expanded from Pancreas Cancer

Panc1-CDB (SEQ ID NO: 14)
DSGVTQTPKHLITATGQRVTLRCSPRSGDLSVYWYQQSLDQ
GLQFLIQYYNGEERAKGNILERFSAQQFPDLHSELNLSSLEL
GDSALYFCASSVQGVSQETQYFGPGTRLVL
Panc5-CD4 A (SEQ ID NO: 15)
DAGVIQSPRHEVTEMGQEVTLRCKPISCHNSLFWYRQTMMR
GLELLIYFNNNVPIDDSGMPEDRFSAKMPNASFSTLKIQPSE
PRDSAVYFCASSRRGSGDTQYFGPGTRLTVL
Panc5-CD4 B (SEQ ID NO:16)
DAGVIQSPRHEVTEMGQEVTLRCKPISGHDYLFWYRQTMMR
GLELLIYFNNNVPIDDSGMPEDRFSAKMPNASFSTLKIQPSE
PRDSAVYFCASSRRGSGDTQYFGPGTRLTVL
Panc7-CD4 B (SEQ ID NO: 17)
KAGVTQTPRYLIKTRGQQVTLSCSPISGHRSVSWYQQTPGQ
GLQFLFEYFSETQRNKGNFPGRFSGRQFSNSRSEMNVSTLEL
GDSALYLCASSTDSNTEAFFGQGTRLTVV
Panc7-CDB B (SEQ ID NO: 18)
DVKVTQSSRYLVKRTGEKVFLECVQDMDHENMFWYRQDPGL
GLRLIYFSYDVKMEKGDIPEGYSVSREKKERFSLILESASTN
QTSMYLCASSFQTAHTDTQYFGPGTRLTVL
Panc 8-CDB (SEQ ID NO: 19)
EAGVTQFPSHSVIEKGQTVTLRCDPISGHDNLYWYRRVMGK
EIKFLLHFVKESKQDESGMPNNRFLAERTGGTYSTLKVQPAE
LEDSGVYFCASSLRDSDEAFFGQGTRLTV
Panc 9-CDB (SEQ ID NO: 20)
NAGVTQTPKFRVLKTGQSMTLLCAQDMNHEYMYWYRQDPGM
GLRLIHYSVGEGTTAKGEVPDGYNVSRLKKQNFLLGLESAAP
SQTSVYFCASSLQGRVDEQFFGPGTRLTVL
Panc 16-CD4 A (SEQ ID NO: 21)
GAVVSQHPSWVICKSGTSVKIECRSLDFQATTMFWYRQFP
KQSLMLMATSNEGSKATYEQGVEKDKFLINHASLTLSTLTVT
SAHPEDSSFYICSARDGTLNTEAFFGQGTRLTW
Panc 16-CD4 B (SEQ ID NO: 22)
GAVVSQHPSWVICKSGTSVKIECRSLDFQATTMFWYRQFP
KQSLMLMATSNEGSKATYEQGVEKDKFLINHASLTLSTLTVT
SAHPEDSSFYICSARPLRDRVAHGYTFGSGTRLTVV
Panc 16-CDB A (SEQ ID NO: 23)
DGGITQSPKYLFRKEGQNVTLSCEQNLNHDAMYWYRQDPGQ
GLRLIYYSQIVNDFQKGDIAEGYSVSREKKESFPLTVTSAQK
NPTAFYLCASSILAFRAGETQYFGPGTRLLVL
Panc 16-CDB B (SEQ ID NO: 24)
GAGVSQSPRYKVTKRGQDVALRCDPISGHVSLYWYRQALGQ
GPEFLTYFNYEAQQDKSGLPNDRFSAERPEGSISTLTIQRTE
QRDSAMYRCASSQGPNYEQYFGPGTRLTVT
Panc 17-CDB A (SEQ ID NO: 25)
GAGVSQSPSNKVTEKGKDVELRCDPISGHTALYWYRQSLGQ
GLEFLIYFQGNSAPDKSGLPSDRFSAERTGGSVSTLTIQRTQ
QEDSAVYLCASSFGLAGANEQFFGPGTRLTVL
Panc 17-CDB B (SEQ ID NO: 26)
GAGVSQSPSNKVTEKGKDVELRCDPISGHTALYWYRQSLGQ
GLEFLIYFQGNSAPDKSGLPSDRFSAERTGGSVSTLTIQRTQ
QEDSAVYLCASSSRLAGTYNEQFFGPGTRLTVL

According to a preferred embodiment the amino acid sequence of the protein according to the invention has sequence identity is at least 95% to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 26. More preferably the sequence identity is at least 98% According to a preferred embodiment the sequence identity to any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 26 is 100%.

The protein may be a T-cell receptor (TCR) in a T-cell obtained by expansion of autologous T-cells. Alternatively, the protein may be a recombinant protein. The methods for producing, i.e. constructing and expressing recombinant proteins are well known in the art (see e.g. (7) and (8)). The recombinant protein may be, for example, designed for introduction into a cell. The cell may be a T-cell, an NK-cell, NK-T-cell or any innate immune cell. Innate immune cells include innate lymphoid cells, MAIT cells or immune memory of effector cells suitable for long-term and effective anti-tumor reactivity.

The recombinant protein may be a transgenic TCR, such as the “anti.NY-ESO-1 TCR” (see e.g. (9)). Such a transgenic TCR may be virally or non-virally transferred into recipient cells. Protocols for the viral and non-viral transfer are known in the art. Examples for the transfer are described in (10), (11) and (12).

According to a second aspect the invention provides a T-cell comprising a TCR of to the first aspect. The T-cell comprising the TCR according to the first aspect is in particular suitable for treatment of cancer. The cancer is preferably selected from acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, mesentery cancer, pancreatic cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colon cancer, and hepatobiliary cancer. The T-cell may preferably be used in the treatment of glioblastoma or pancreatic cancer.

As shown in the examples, T-cell products comprising a TCR with a Vbeta sequence selected from any of SEQ ID NO: 1 to 26 are able to recognize and kill tumor cells in vitro. Therefore, identification of said TCR in a T-cell product drastically improves the chance that the T-cell product is able to fight the cancer in vivo, in particular that the T-cell product is clinically/biologically relevant.

Thus, according to a third aspect the invention provides a method of selecting a T-cell product for use in active immunotherapy in a patient, wherein the method comprises the following steps:
a) providing at one or more T-cell products;
b) identifying the presence of a according to the first aspect in the one or more T-cell products; and
c) selecting the T-cell product comprising a TCR identified in step b) for use in active immune therapy.

According to one embodiment, the method further comprises determining the clonality of the Vβ-family to which the TCR belongs and selecting the T-cell product in which the TCR is present and the Vβ-family to which the TCR belongs is monoclonal. Monoclonality of the TCR indicated a directed and specific expansion of the TCR.

According to one embodiment the method further comprises determining the percentage of the TCR and/or the Vβ family to which the TCR belongs in the T-cell product and selecting the T-cell product in which the percentage of the TCR is the highest. In order to provide clinical/biological relevance to the T-cell product the TCR should be present in a sufficient concentration.

The method according to third aspect may be used in particular in the treatment of cancer. Thus, the method is preferably carried out with a patient suffering from cancer. The cancer may be, for example, one of the cancers listed above. Preferably the cancer is selected from glioblastoma and pancreas cancer.

In case of a patient with a glioblastoma, T-cell products containing a TCR with a sequence identity to any of the sequences SEQ ID NO: 1-13 is preferred. In case of a patient with pancreatic cancer T-cell products containing a TCR with a sequence identity to any of the sequences SEQ ID NO: 1-13 is preferred.

According to one embodiment of the third aspect providing the one or more T-cell products is based on an expansion of T-cells according to the invention. More preferably the method comprises
providing a body sample containing T-cells from the patient;
culturing cells of the sample in-vitro in the presence of at least one cytokine.

The body sample can derived from any part of the body that contains lymphocytes. Examples of body samples are peripheral blood, cord blood, bone marrow, lymph nodes, liver pleural effusion, thorax, abdominal cavity, synvial fluid, peritoneum, retroperitoneal space, thymus, and tumor.

Preferably, the step of providing the body sample does not involve a step of interference, in particular treatment or surgery of the human body.

Preferably the culturing for expansion of the T-cells occurs in the presence of IL-2, IL-15 and IL-21. According to a further embodiment, the concentration of IL-2 in the liquid composition is in the range of from 10 to 6000 U/ml. The International Unit (U) is the standard measure for an amount or IL-2. It is determined by its ability to induce the proliferation of CTLL-2 cells. The concentration of IL-2 is preferably in the range from 500 to 2000 U/ml. More preferably the concentration of IL-2 is in the range from 800 to 1100 U/ml. According to one embodiment the concentration of IL-15 is in the range of 0.1 to 100 ng/ml. Preferably, the concentration of IL-15 is in the range from 2 to 50 ng/ml, more preferably in the range from 5 to 20 ng/ml. The most preferred concentration is about 10 ng/ml. In a further embodiment the concentration of IL-21 is in the range from 0.1 ng/ml, preferably in the range from 2 to 50 ng/ml, more preferably in the range from 5 to 20 ng/ml.

According to one embodiment of the invention the culture medium of the first expansion step comprises at least one expansion antigen. The expansion antigen is a known disease associated antigen or a fragment, a mutant or a variant thereof. As used herein a “mutant” is defined as an amino acid sequence that differs from the reference sequence by an insertion, deletion or replacement of at least one amino acid. The expansion antigen is a TAA.

According to a preferred embodiment of the method according to the second aspect, the body sample is tumor and no expansion antigen is used in culturing.

The invention is further defined by the following examples.

### EXAMPLES

### Example 1—Expansion of TIL and Tumor Cell Culture from Glioma Patients

1.1 Diagnosis and Patients:

16 patients (Supplementary Table 1) with gliomas were enrolled. The study was approved by the regional ethical review board at Karolinska Institutet, Stockholm, Sweden (Dnr: 2013/576-31).

1.2 TIL Expansion:

Glioma tumor tissue was harvested in the course of tumor surgery and dissected into fragments (approximately 1-2mm³) using a sterile scalpel. The fragments were washed 2 times with PBS and cultured in 24 well plates in GMP Serum-free DC medium (CellGenix, Freiburg, Germany) plus 5% pooled human AB serum (Innovative Research, Michigan, USA) supplemented with recombinant IL-2 (1000 IU/ml), IL-15 (10 ng/ml), IL-21 (10 ng/ml) (Prospec, Ness-Ziona, Israel). The culture medium was changed when necessary. TILs were transferred into 6 well plates; as they covered >70% of the 24 well surface, they were further expanded in G-Rex flasks (Wilson Wolf, New Brighton, USA) using 30ng OKT3/mL and irradiated allogeneic feeder cells at the ratio of 1 feeder cell to 10 TILs.

1.3 Tumor Cell Generation

Tumor single cell suspension were processed using a Medimachine (BD, California, USA Cat:340588). The cell suspension was washed 2 times in PBS and then cultured in T25 flasks (Thermo Fisher Scientific Inc., Waltham, Mass. USA) using RPMI 1640 L-glutamine 2mM (Life Technologies, CA, USA) supplemented with antibiotics plus 20% Fetal Bovine Serum (Gibco, REF: 26140-079).

### Example 2—Exposure of TIL to Autologous Tumor Cell Co-Cultures.

It was tested whether the expanded TIL recognized the autologous tumor cell line defined by intracellular cytokine production. For this, expanded TILs were exposed to autologous tumor cells in 96 well plates. Cells were seeded at 20,000 TILs/well at an E:T ratio of 10:1. E:T is the ratio of expanded TIL to tumor cells. Supernatants were harvested at day 3 and tested for IFNγ by Elisa (MABTECH, Stockholm, Sweden).

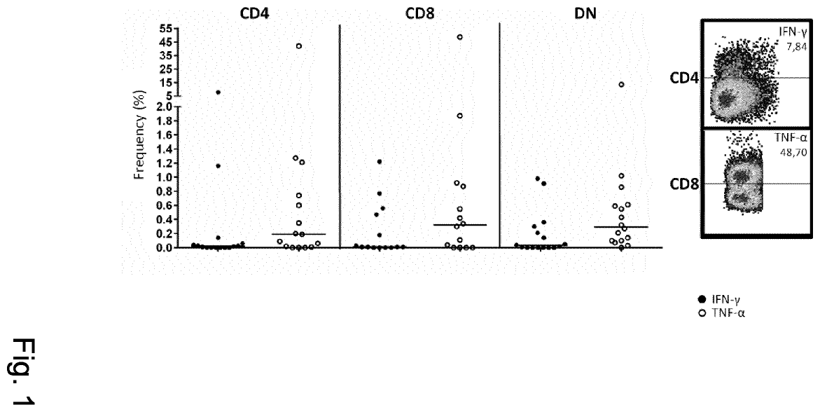

A IFNγ and TNFα production against the autologous tumor cells in expanded TIL from individual patients up to 7.87% in CD3+CD4+ and up to 48.70% in CD3+CD8+TIL was observed (see FIG. 1)

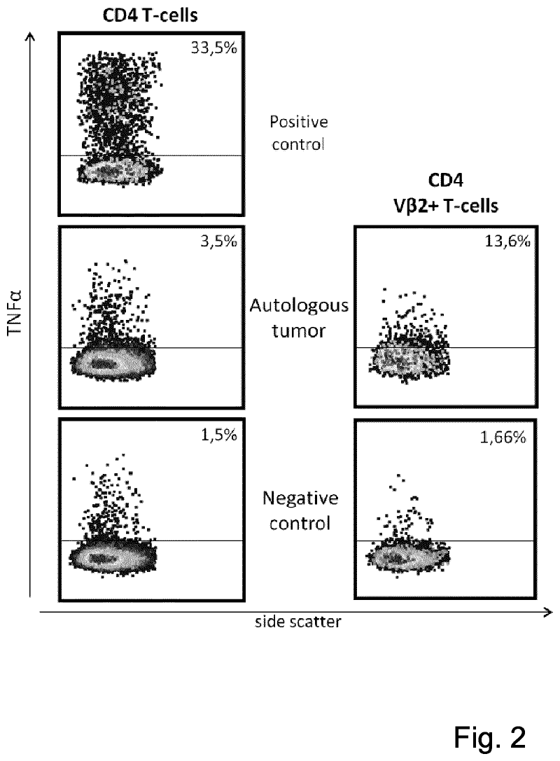

The cytokine production from GBM-D CD3+CD4+ TCRVβ2 T-cells (representing 33.3% TCRV(32 T-cells in CD3+CD4 TIL) (FIG. 2). CD3+CD4+ TIL exhibited TNFα production of 3.5% in response to autologous tumor cells, its TCRVβ2 T-cell subpopulation showed a higher frequency (1.,6%.) of TNFα producing CD3+CD4+ cells. Testing of TIL reactivity in ICS also revealed selective IL-2 and IL-17 production of individual TIL lines against the autologous tumor cells.

### Example 3—Link of the Individual TCR Vβ Families with Tumor Recognition

TCR Vβ Families were Analyzed by Flow Cytometry and PCR.

3.1 Vβ Family Analysis by Flow cytometry

TCR Vβ frequency staining was performed using the Beta Mark TCR Vβ Repertoire Kit (Beckman Coulter, CA, USA) along with co-staining with anti-CD3 PE-Cy7 (BD Biosciences, CA, USA), anti-CD4 Krome Orange (Beckman coulter, CA, USA) and anti-CD8a APC-Cy7 (BD Biosciences, CA, USA). After washing, a FACS Aria flow cytometer (BD Biosciences, Stockholm, Sweden) was used for acquisition and data analysis was performed by FlowJo software. TCR CDR3 analysis was performed using the TCR Vβ panel as described before (Magalhaes et al., 2008). In brief, TILs were sorted using CD4+ or CD8+ magnetic beads (MACS Milteny Biotec AB, Lund, Sweden) according to the supplier's instructions.

3.2 Vβ Family Analysis by PCR

For the PCR analysis, total RNA from CD4+ and CD8+ positive sorted cells was extracted using a RNeasy plus RNA extraction kit (Qiagen inc. Hilden, Germany) and converted to cDNA using a Oligodt protocol from revertaid premium first strand cDNA synthesis kit (Fermentas, ThermoFisher scientific, MA, USA). The multiplex PCR was performed using Qiagen multiplex PCR kit in a 30 μl reaction volume. Each reaction contained 15 μl of Qiagen multiplex PCR mastermix, 20 pmol of the respective forward primer mix (primer mix 1 to 9), 20 pmol of 6FAM tagged TCRVβ content reverse primer (Ahmed et al., 2014).

A positive control reaction was performed for each sample using primers covering constant region of TCR Vβ gene specific primers tagged with VIC fluorochrome. The PCR was initiated with denaturation at 95° C. for 15 min followed by 35 cycles of 94° C. for 30 sec, 61° C. for 90 sec and 72° C. for 90 sec. The reaction was terminated at 72° C. for 10 min. The analysis was performed on ABI7500 capillary electrophoresis fragment analyzer (Applied Biosystems, CA, USA) using 400 bp internal control ladder for size reference.

Samples exhibiting monoclonal expansion of T-cells were amplified with family specific primers and cloned into a TA (PCR2.1 topo) vector (Invitrogen, CA, USA) according to the suppliers instructions. The inserts in the plasmids were sequenced to identify the TCR CDR3 region using BigDye terminator cycle sequencing kit and ABI7500 capillary electrophoresis instrument (Applied Biosystems, CA, USA).

3.2 Results

The analyzed T-cell products showed up to 99% of individual Vβ families in TIL from individual patients (see Table 3), e.g. the TCRVβ2 which represented 99,6% in CD3+CD4+ T-cells from the GBM-I TIL, the TCRV65.1 family constituted 97% of GBM-I CD3+CD8+ T-cells; TCRVβ21.3 constituted 98.4% of the GBM-J CD3+CD4+ T-cells. Some of the dominant TCR Vβ families were analyzed in greater detail and tested for the CDR3 length, followed by sequencing of the TCR, i.e. some of the expanded TCRVβ (see Table 4) represent T-cell clones.

TABLE 3
TCR Vβ CDR3 analysis

| | GBM-A | | GBM-B | | GBM-C | | GBM-D | | GBM-E | |
|---|---|---|---|---|---|---|---|---|---|---|
| Patient | CD4 | CD8 | CD4 | CD8 | CD4 | CD8 | CD4 | CD8 | CD4 | CD8 |
| Vβ1 | 0.24 | 0.74 | 2.45 | 0.74 | 1.49 | 11.00 | 0.19 | 0.12 | 4.06 | 0.25 |
| Vβ2 | 34.3 (1) | 0.11 | 17.60 | 2.52 | 7.92 | 3.69 | 33.3 (O) | 1.67 | 13.60 | 2.48 |
| Vβ3 | 3.54 | 0.36 | 2.97 | 9.29 | 6.83 | 10.30 | 1.33 | 1.00 | 9.81 | 2.11 |
| Vβ4 | 0.14 | 0.03 | 0.27 | 0.04 | 0.04 | 0.01 | 0.05 | 0.07 | 0.07 | 0.03 |
| Vβ5.1 | 0.34 | 0.18 | 1.72 | 0.76 | 0.18 | 0.11 | 0.14 | 0.09 | 9.98 | 0.11 |
| Vβ5.2 | 1.63 | 0.10 | 0.65 | 0.33 | 0.65 | 0.87 | 0.23 | 2.23 | 0.40 | 0.16 |
| Vβ5.3 | 0.07 | 0.23 | 1.01 | 0.82 | 0.42 | 1.48 | 0.03 | 0.08 | 0.64 | 0.02 |
| Vβ7.1 | 1.91 | 0.11 | 0.86 | 0.38 | 2.00 | 4.99 | 0.07 | 1.80 | 1.28 | 0.03 |
| Vβ7.2 | 3.73 | 1.53 | 1.53 | 0.86 | 1.19 | 0.32 | 0.35 | 6.80 | 0.97 | 0.03 |
| Vβ8 | 2.21 | 1.67 | 6.90 | 3.25 | 4.89 | 1.23 | 2.34 | 1.23 | 6.71 | 0.25 |
| Vβ9 | 0.90 | 0.01 | 0.56 | 0.53 | 2.74 | 0.14 | 0.28 | 1.00 | 0.18 | 5.66 |
| Vβ11 | 0.25 | 0.15 | 0.12 | 2.17 | 0.95 | 2.64 | 0.43 | 0.42 | 0.85 | 0.05 |
| Vβ12 | 2.37 | 28.4 (O) | 4.18 | 2.34 | 4.44 | 2.20 | 3.67 | 0.46 | 12.10 | 1.04 |
| Vβ13.1 | 0.88 | 3.26 | 2.60 | 1.12 | 5.27 | 1.14 | 4.47 | 2.08 | 0.41 | 0.06 |
| Vβ13.2 | 0.35 | 0.03 | 3.25 | 2.02 | 1.06 | 2.95 | 0.88 | 0.56 | 0.59 | 0.10 |
| Vβ13.6 | 2.14 | 0.19 | 1.64 | 0.15 | 1.84 | 0.60 | 0.56 | 0.74 | 3.01 | 0.05 |
| Vβ14 | 0.76 | 4.47 | 3.63 | 3.87 | 6.75 | 6.03 | 2.00 | 3.38 | 3.87 | 1.98 |
| Vβ16 | 2.90 | 0.70 | 3.84 | 1.97 | 4.28 | 5.27 | 17.7 (N) | 4.38 | 2.27 | 1.70 |
| Vβ17 | 0.35 | 0.03 | 1.35 | 0.28 | 4.71 | 0.00 | 0.11 | 1.25 | 0.83 | 0.27 |
| Vβ18 | 0.08 | 0.08 | 0.62 | 0.10 | 0.21 | 0.08 | 0.39 | 0.04 | 0.80 | 0.03 |
| Vβ20 | 16.7 (N) | 0.45 | 10.50 | 2.92 | 8.16 | 4.39 | 3.67 | 2.88 | 2.68 | 0.31 |
| Vβ21.3 | 0.41 | 41.5 (2) | 2.97 | 3.71 | 3.61 | 8.54 | 4.18 | 6.71 | 2.47 | 45.90 |
| Vβ22 | 26.80 | 0.06 | 0.70 | 0.00 | 1.63 | 1.63 | 1.33 | 35.4 (O) | 6.50 | 0.02 |
| Vβ23 | 1.39 | 0.07 | 0.79 | 0.53 | 1.20 | 2.56 | 0.10 | 0.09 | 0.91 | 0.02 |

| | GBM-F | | GBM-G | | GBM-H | | GBM-I | |
|---|---|---|---|---|---|---|---|---|
| Patient | CD4 | CD8 | CD4 | CD8 | *CD4 | CD8 | CD4 | CD8 |
| Vβ1 | 0.09 | 10.30 | 0.00 | 0.00 | 0.19 (2) | 0.06 | 0.01 | 0.00 |
| Vβ2 | 0.17 | 0.00 | 0.54 | 0.00 | 1.21 (1) | 0.00 | 99.6 (2) | 0.00 |
| Vβ3 | 9.21 | 0.00 | 0.00 | 0.00 | 0.92 | 0.00 | 0.00 | 0.00 |
| Vβ4 | 0.34 | 0.00 | 0.00 | 0.00 | 0.16 | 0.09 | 0.08 | 0.00 |
| Vβ5.1 | 0.06 | 0.00 | 0.00 | 0.00 | 0.24 | 0.00 | 0.00 | 97 (2) |
| Vβ5.2 | 0.56 | 0.00 | 0.07 | 0.00 | 0.71 | 0.25 | 0.04 | 0.00 |
| Vβ5.3 | 0.05 | 0.00 | 0.01 | 0.00 | 0.08 | 0.00 | 0.00 | 0.00 |
| Vβ7.1 | 0.08 | 0.00 | 0.00 | 0.00 | 0.64 | 0.00 | 0.01 | 0.00 |
| Vβ7.2 | 0.13 | 0.00 | 0.00 | 0.00 | 0.00 | 0.00 | 0.09 | 0.00 |
| Vβ8 | 1.10 | 0.00 | 0.03 | 0.00 | 4.77 (1) | 40.4 (2) | 0.01 | 0.65 |
| Vβ9 | 0.04 | 0.00 | 0.04 | 2.86 | 0.39 | 0.11 | 0.00 | 0.27 |
| Vβ11 | 0.02 | 0.00 | 0.01 | 0.00 | 0.19 | 0.07 | 0.07 | 0.00 |
| Vβ12 | 0.03 | 0.00 | 0.02 | 0.00 | 0.14 | 0.00 | 0.06 | 0.00 |
| Vβ13.1 | 0.02 | 0.00 | 85.1 (1) | 0.00 | 1.08 | 0.08 | 0.00 | 0.00 |
| Vβ13.2 | 0.71 | 8.70 | 0.10 | 3.57 | 0.08 | 0.27 | 0.01 | 0.00 |
| Vβ13.6 | 0.12 | 0.00 | 0.37 | 0.00 | 0.32 | 0.08 | 0.00 | 0.00 |
| Vβ14 | 48.2 (O) | 5.26 | 0.01 | 0.00 | 1.68 | 45.3 (2) | 0.01 | 0.00 |
| Vβ16 | 0.24 | 0.00 | 0.00 | 0.00 | 0.21 | 0.05 | 0.01 | 0.00 |
| Vβ17 | 0.14 | 0.00 | 0.00 | 42.9 (N) | 10.20 | 0.00 | 0.02 | 0.00 |
| Vβ18 | 0.01 | 0.00 | 0.01 | 0.00 | 0.00 | 0.00 | 0.00 | 0.00 |
| Vβ20 | 0.08 | 0.00 | 8.48 | 2.04 (1) | 3.62 | 0.06 | 0.00 | 0.43 |
| Vβ21.3 | 3.34 | 3.45 | 0.01 | 0.00 | 0.13 | 0.50 | 0.01 | 0.00 |
| Vβ22 | 0.96 | 0.00 | 0.01 | 0.00 | 0.46 (1) | 9.96 | 0.01 | 0.00 |
| Vβ23 | 0.05 | 0.00 | 0.12 | 0.00 | 5.42 | 0.19 | 0.00 | 0.00 |

| | GBM-J | GBM-K | | GBM-L | | GBM-M | | GBM-N | | GBM-O | | GBM-P | |
|---|---|---|---|---|---|---|---|---|---|---|---|---|---|
| Patient | CD4 | *CD4 | *CD8 | CD4 | CD8 | *CD4 | CD8 | CD4 | CD8 | CD4 | CD8 | CD4 | CD8 |
| Vβ1 | 0.02 | 0.58 (1) | 0 (1) | 1.72 | 1.78 | 0.47 | 1.72 | 0.27 | 0.28 | 1.37 | 3.70 | 0.13 | 0.04 |
| Vβ2 | 0.06 | 0.25 (1) | 0.029 (1) | 12.20 | 0.90 | 1.39 | 3.72 | 40.90 | 0.04 | 2.38 | 2.30 | 1.24 | 0.02 |
| Vβ3 | 0.00 | 0.81 (1) | 0.12 (1) | 4.46 | 0.38 | 19.30 | 3.70 | 1.33 | 0.02 | 1.34 | 3.09 | 0.12 | 0.20 |
| Vβ4 | 0.00 | 0.11 | 0.00 | 0.47 | 0.08 | 0.15 | 0.98 | 0.03 | 0.01 | 0.01 | 0.08 | 0.01 | 0.01 |
| Vβ5.1 | 0.01 | 0.39 | 0.37 | 7.32 | 1.67 | 0.08 | 0.00 | 0.33 | 0.45 | 0.16 | 0.26 | 0.29 | 0.18 |
| Vβ5.2 | 0.32 | 0.43 | 0.06 | 0.47 | 0.61 | 0.15 | 0.53 | 1.28 | 0.10 | 0.27 | 0.80 | 0.26 | 0.18 |
| Vβ5.3 | 0.00 | 0.09 | 0.00 | 2.91 | 4.48 | 0.22 | 0.85 | 0.05 | 0.02 | 0.06 | 0.13 | 0.06 | 1.37 |
| Vβ7.1 | 0.00 | 0.21 | 0.00 | 1.12 | 1.51 | 0.65 | 1.99 | 5.07 | 90.70 | 0.05 | 0.25 | 0.15 | 0.37 |
| Vβ7.2 | 0.01 | 0.97 | 0.04 | 2.71 | 6.92 | 0.05 | 2.93 | 0.11 | 0.00 | 0.95 | 1.39 | 0.01 | 0.04 |
| Vβ8 | 0.00 | 3.02 | 2.65 | 4.88 | 2.35 | 3.41 | 23.10 | 1.02 | 0.07 | 0.28 | 0.22 | 0.13 | 0.23 |
| Vβ9 | 0.00 | 0.02 | 0.00 | 2.19 | 0.72 | 1.70 | 3.55 | 7.62 | 0.07 | 0.31 | 2.97 | 7.98 | 0.16 |
| Vβ11 | 0.01 | 0.29 | 0.04 | 2.82 | 0.69 | 0.19 | 0.43 | 0.10 | 0.10 | 0.13 | 0.30 | 0.21 | 1.63 |
| Vβ12 | 0.00 | 0.53 | 0.00 | 2.60 | 0.34 | 1.93 | 6.91 | 1.81 | 0.04 | 0.51 | 11.60 | 0.09 | 0.02 |
| Vβ13.1 | 0.03 | 0.09 | 0.06 | 15.50 | 0.62 | 11.10 | 1.54 | 0.18 | 0.05 | 3.15 | 2.44 | 23.90 | 0.03 |
| Vβ13.2 | 0.01 | 6.41 | 0.38 | 3.14 | 6.70 | 19.90 | 1.95 | 0.87 | 0.27 | 0.18 | 2.56 | 0.41 | 1.58 |
| Vβ13.6 | 0.00 | 0.16 | 0.15 | 2.71 | 6.54 | 1.08 | 3.08 | 0.09 | 0.27 | 0.15 | 0.41 | 1.34 | 0.03 |
| Vβ14 | 0.00 | 13.7 (N) | 0.77 | 2.72 | 1.80 | 1.91 | 18.70 | 0.13 | 0.01 | 2.48 | 2.84 | 0.57 | 0.28 |
| Vβ16 | 0.02 | 1.59 | 0.06 | 5.29 | 4.17 | 0.97 | 5.08 | 0.16 | 0.04 | 0.23 | 1.92 | 1.33 | 0.17 |
| Vβ17 | 0.00 | 0.06 | 0.03 | 2.91 | 1.94 | 0.83 | 1.52 | 0.40 | 0.06 | 0.06 | 21.80 | 56.00 | 0.07 |
| Vβ18 | 0.00 | 0.34 | 0.09 | 3.17 | 7.08 | 0.01 | 0.00 | 0.03 | 0.05 | 0.12 | 0.26 | 0.24 | 0.15 |
| Vβ20 | 1.01 | 1.1 (2) | 0.46 (1) | 4.84 | 1.32 | 0.10 | 0.00 | 0.36 | 0.01 | 0.12 | 1.15 | 0.18 | 0.05 |
| Vβ21.3 | 98.4 (2) | 5.06 | 0.04 | 1.15 | 36.40 | 0.23 | 23.30 | 0.08 | 0.06 | 0.20 | 1.06 | 9.50 | 0.07 |
| Vβ22 | 0.10 | 1.01 | 0.21 | 1.13 | 1.09 | 0.30 | 0.00 | 0.04 | 0.11 | 0.22 | 0.40 | 0.06 | 7.30 |
| Vβ23 | 0.00 | 1.39 | 0.20 | 2.86 | 3.82 | 0.36 | 0.86 | 0.40 | 0.58 | 0.83 | 5.83 | 0.37 | 0.16 |

TABLE 4
Vβ amino acid sequences

| | | | | | | Flow |
|---|---|---|---|---|---|---|
| | | | | | | anti- |
| TIL id | Vβ | Vβ | (-D-) | Jβ | Jβ | body |
| GBM A- | TRBV20- | CSA | ATGDRP | YEQYF | TRBJ2-7 | Vβ2 |
| CD4 | 1 | | | | | |
| GBM A- | TRBV10- | CAI | RTGSD | NEQSF | TRBJ2-1 | Vβ12 |
| CD8 | 3 | | | | | |
| GBM A- | TRV11 | CAS | RYTGS | IEQFF | TRBJ2-1 | Vβ21.3 |
| CD8 | | | | | | |
| GBM F- | TRBV27 | CAS | SAGTSG | YEQYF | TRBJ2-7 | Vβ14 |
| CD4 | | | VT | | | |
| GBM G- | TRBV6- | CAS | STR | FEQYF | TRBJ2-7 | Vβ13.1 |
| CD4 | 1/5/6 | | | | | |
| GBM H- | TRBV12- | CAS | SSRRDH | NGQFF | TRBJ2-1 | Vβ8 |
| CD8 | 3/4 | | TY | | | |
| GBM H- | TRV27 | CAS | SLQGAN | YGYTF | TRBJ1-2 | Vβ14 |
| CD8 | | | RVIPSGGV | | | |
| GBM I- | TRBV20- | CSA | VQGT | DTQYF | TRBJ2-3 | Vβ2 |
| CD4 | 1 | | | | | |
| GBM I- | TRBV5-1 | CAS | SWDKS | YEQYF | TRBJ2-7 | Vβ5.1 |
| CD8 | | | | | | |
| GBM J- | TRBV11 | CAS | SRLALFS | YEQYF | TRBJ2-7 | Vβ21.3 |
| CD4 | | | | | | |

### Example 4—Expansion of TIL and Tumor Cell Culture from Pancreas Cancer Patients

4.1 Diagnosis and Patients.

17 patients (Table 1) with pancreatic cancer (13/17 with a ductal adenocarcinoma), recruited at the Pancreatic Surgery Unit of the Karolinska Hospital, Huddinge, Sweden, provided informed consent. The study was approved by the regional ethical review board at Karolinska Institutet, Stockholm, Sweden (Dnr: 2013/576-31).

4.2 Initial TIL culture

Tumor tissues from clinical biopsies or surgery were cut with surgical scissors and a scalpel. Individual single tumor fragments (1-2 mm³) were placed in each well of a 24-well tissue culture plate (Costar, USA) along with 1 ml of TIL medium , i.e. Cellgro GMP Serum-free medium (CellGenix, Freiburg, Germany), with 10% human AB serum (Innovative Research, Michigan, USA), supplemented with IL-2 1000 IU/ml, IL-15 10 ng/ml, IL-21 10 ng/ml, (Prospec, Ness-Ziona, Israel), penicillin (100 IU/mL), streptomycin (100 ·g/mL) (Life Technologies, Carlsbad, USA) and Amphotericin B (2.5 mg/L) (Sigma-Aldrich, MI; USA). Plates were then incubated at 37° C., 5% CO₂. On day 4, 1 ml medium was added to wells. On day 7, TIL were split into 2-3 additional 24-wells. Upon 80% expansion of TIL in each culture well, 1 ml of TIL medium was added.

4.3 TIL Expansion Protocol.

TIL were further expanded on day 10 using OKT3 (Biolegend, CA, USA) in the presence of a 1:10 ratio of 55 Gry irradiated allogeneic feeder cells from healthy volunteers, resuspended in medium (Cellgro with 10% human AB serum). OKT3 was used at 30 ng/ml along with IL-2 1000 IU/ml, IL-15 10 ng/ml, and IL-21 10 ng/ml. TIL were then transferred to 6-well plates and tested for anti-tumor (as autologous tumor cells were available) or TAA-reactivity. If tested positive, defined by IFN·production, they were expanded in GRex flasks (Wilson Wolf, New Brighton, Minn., USA) using TIL medium (Cellgro with 10% human AB serum, IL-2 1000 IU/ml, IL-15 10 ng/ml, and IL-21 10 ng/m.) plus irradiated (55Gy) feeder cells (ratio of feeder cells: TIL was 1:10); OKT3 was used at 30 ng/ml.

4.4 Tumor Cell Generation

After surgery or biopsy, tumor tissues were cut with surgical scissors and a scalpel. Single tumor fragments (1-2 mm3) were placed in 24-well tissue culture plates with 1 ml of tumor medium, i.e. RPMI 1640 with 10% FBS (Life technologies, CA, USA), Epidermal growth factor (20 ng/ml, ImmunoTools, Friesoythe, Germany) supplemented with antibiotics (penicillin,100 IU/mL and streptomycin, 10 mg/mL) (Life Technologies, Carlsbad, USA) and Amphotericin B (2.5 mg/L, Sigma-Aldrich, MI; USA). Tumor cell lines were then cultured and passed without EGF.

### Example 5—Pancreas Cancer: Exposure of TIL to Autologous Tumor Cell Co-Cultures

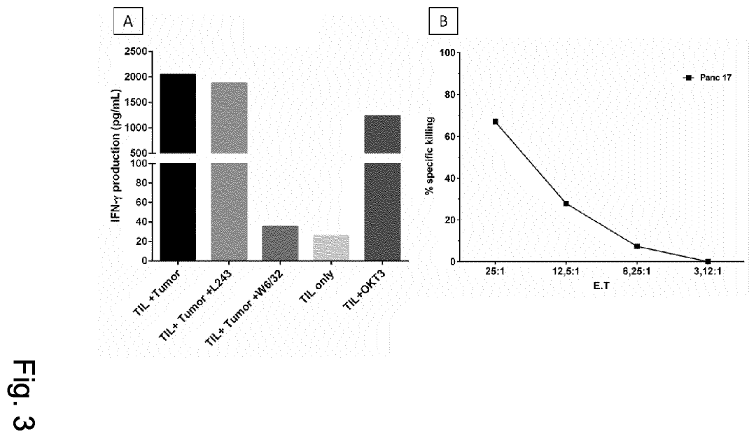

The autologous tumor cell line established from Panc 17 was tested for TIL recognition (Table 5). IL-2 and IL-17 production tested positive in CD3+CD4+ TIL (1.55%). CD3+CD4−CD8− (DN) T-cells showed IL-2 (3.71%) and IL-17 (9.76%) production in response to autologous tumor cells. The same TIL line showed strong cytotoxicity directed against tumor cells, tested by a standard Cr51 release assay (see FIG. 3A).

The Panc 9 TIL (99.2% CD8+ Vβ13.2, a monoclonal TCR see supplementary Table S2) exhibited IFN-γ production against freshly harvested autologous tumor single cell suspension cells, i.e. 2022.81pg/ml (see FIG. 3B), which could be blocked using the anti-MHC-I antibody (W6/32, 9.91 pg/ml); the anti-HLA-DR antibody (L243) did not affect IFN-γ production (1853.45 pg/ml).

### Example 6—TCRVβ CDR3 Analysis

We analyzed the TCR Vβ families in TIL with a preferential expansion of individual Vβ families.

6.1 TCRVβ CDR3 Analysis by Flow cytometry

TCR Vβ frequency staining was performed using the Beta Mark TCR Vβ Repertoire Kit (Beckman Coulter, France) along with CD3 PE-Cy7 (BD Biosciences, CA, USA), CD4 Krome orange (Beckman Coulter, France), and CD8 APC-Cy7(BD Biosciences, CA, USA). TIL were washed using PBS-0.1%FBS and stained using a FACS Aria flow cytometer (BD Biosciences, Stockholm, Sweden).

6.2 TCRVβ CDR3 Analysis by PCR:

TCR CDR3 analysis was performed using the TCR Vβ panel as described before [29]. The TCR Vβ CDR3 analysis was performed using the TCR Vβ family specific primer set panel previously described. First, TILs were sorted using CD4+ or CD8+ magnetic beads (MACS Miltenyibiotec AB, Lund, Sweden) according to the supplier's instructions. Total RNA from CD4 and CD8 positive sorted cells was extracted using a RNeasy plus RNA extraction kit (Qiagen inc. Hilden, Germany) and converted to cDNA using a Oligodt protocol from the revertaid premium first strand cDNA synthesis kit (Fermentas, ThermoFisher scientific, MA, USA). The multiplex PCR was performed using Qiagen multiplex PCR kit in a 30·|reaction volume. Each reaction contained 15·|of the Qiagen multiplex PCR mastermix, 20 pmol of the respective forward primer mix (primer mix 1 to 9) and 20 pmol of 6FAM tagged TCRVβ content reverse primer. A positive control reaction was performed for each sample using primers covering constant region of TCR Vβ gene specific primers tagged with VIC fluorochrome. The PCR was initiated with denaturation at 95° C. for 15 min followed by 35 cycles of 94° C. for 30 seconds, 61° C. for 1 minute 30 seconds and 72° C. for 1 minute 30 seconds. The reaction was terminated at 72° C. for 10 minutes. Analysis was performed on ABI7500 capillary electrophoresis fragment analyzer (Applied Biosystems, CA, USA) using an 400bp internal control ladder for size reference. Samples exhibiting strong monoclonal expansion of T-cells were amplified with family specific primers and cloned into a TA (PCR2.1 topo) vector (Invitrogen, CA, USA) according to the suppliers instructions. The inserts in the plasmids were sequenced to identify the TCR CDR3 region using BigDye terminator cycle sequencing kit and the ABI7500 capillary electrophoresis instrument (Applied Biosystems, CA, USA).

6.3 Results

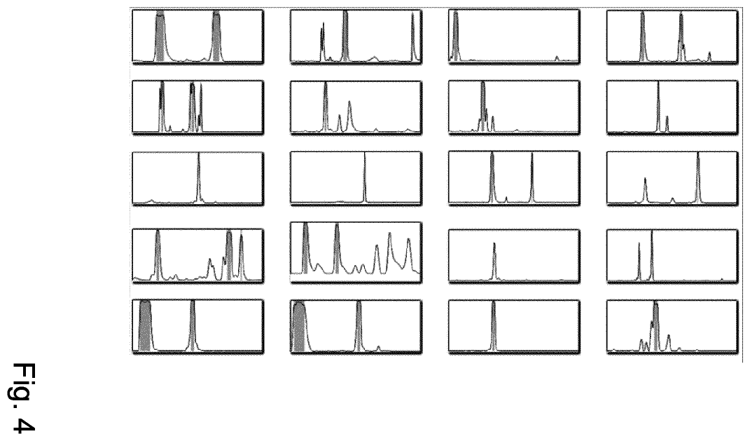

Panc 9 (CD8+) TIL exhibited up to 99.2% Vβ13.2+ T-cells. The preferential expansion of TCR Vβ families was also observed in other TIL lines (see Table 5), e.g. CD8⁺ Vβ1 (77.1%) from Panc1. CD4⁺ Vβ8 (42.7%) in Panc5, CD8⁺ Vβ2 (39.8%) in Panc10, CD4⁺ Vβ2 (48.9%) in Panc16. Some of the expanded Vβ families showed clonal TCRs tested by CDR3 TCR length analysis, followed by TCR sequencing (see Tables 6 & 7 and FIG. 4).

TABLE 5
Vβ analysis in TILs. Numbers indicated the frequency of TCR Vbeta families in CD4+ and CD8+ TIL

| | Panc 1 | | Panc 2 | | Panc 3 | | Panc 4 | | Panc 5 | | Panc 6 | | Panc 7 | | Panc 8 | | Panc 9 | |
|---|---|---|---|---|---|---|---|---|---|---|---|---|---|---|---|---|---|---|
| | CD4 | CD8 | CD4 | CD8 | CD4 | CD8 | CD4 | CD8 | CD4 | CD8 | CD4 | CD8 | CD4 | CD8 | CD4 | CD8 | CD4 | CD8 |
| Vβ1 | 2.8 | 77.1 | 3.2 | 2.3 | 3.4 | 0.8 | 0.2 | 2.1 | 1.5 | 0.2 | 0.7 | 1.1 | 0.3 | 0.1 | 1.5 | 1.2 | 0.6 | 0.1 |
| Vβ2 | 8.0 | 0.6 | 9.8 | 2.6 | 4.8 | 0.2 | 8.5 | 2.0 | 0.0 | 0.0 | 9.6 | 19.9 | 0.1 | 0.7 | 7.4 | 1.7 | 12.0 | 0.0 |
| Vβ3 | 2.1 | 0.2 | 4.8 | 1.9 | 26.7 | 0.5 | 4.8 | 8.3 | 0.0 | 6.1 | 4.2 | 0.3 | 5.1 | 28.7 | 2.4 | 12.0 | 1.0 | 0.0 |
| Vβ4 | 0.4 | 0.0 | 0.0 | 0.0 | 0.1 | 0.0 | 0.0 | 0.0 | 0.0 | 0.0 | 0.0 | 0.0 | 0.1 | 0.0 | 0.1 | 0.0 | 3.2 | 0.0 |
| Vβ5.1 | 0.4 | 0.4 | 0.7 | 0.5 | 0.3 | 0.0 | 0.1 | 0.0 | 0.0 | 0.3 | 9.0 | 0.3 | 41.1 | 6.2 | 9.3 | 0.0 | 3.6 | 0.1 |
| Vβ5.2 | 0.2 | 0.1 | 1.5 | 0.0 | 1.5 | 0.0 | 0.1 | 0.2 | 0.2 | 0.0 | 0.2 | 13.7 | 2.4 | 0.2 | 0.3 | 0.2 | 0.1 | 0.1 |
| Vβ5.3 | 0.1 | 0.1 | 1.6 | 0.4 | 1.9 | 0.0 | 0.0 | 0.0 | 0.0 | 0.0 | 0.3 | 0.1 | 0.0 | 0.9 | 0.6 | 0.1 | 0.0 | 0.0 |
| Vβ7.1 | 0.2 | 0.3 | 0.7 | 2.1 | 0.9 | 3.4 | 0.2 | 0.1 | 0.1 | 0.0 | 0.1 | 0.1 | 0.1 | 0.2 | 5.1 | 0.3 | 0.6 | 0.0 |
| Vβ7.2 | 6.6 | 0.0 | 0.3 | 0.1 | 0.9 | 1.7 | 0.2 | 0.1 | 0.0 | 0.0 | 1.1 | 0.1 | 1.0 | 0.4 | 5.3 | 3.8 | 13.9 | 0.0 |
| Vβ8 | 1.9 | 1.7 | 3.7 | 0.6 | 2.9 | 0.1 | 0.4 | 0.4 | 42.7 | 4.1 | 1.2 | 0.3 | 5.4 | 0.5 | 2.3 | 2.2 | 0.5 | 0.1 |
| Vβ9 | 1.6 | 0.0 | 0.8 | 0.3 | 0.3 | 0.1 | 0.1 | 0.1 | 40.5 | 0.1 | 1.7 | 0.0 | 0.2 | 0.1 | 1.0 | 0.1 | 4.8 | 0.0 |
| Vβ11 | 0.5 | 0.0 | 0.5 | 0.2 | 1.5 | 0.0 | 0.1 | 0.7 | 0.3 | 0.0 | 0.6 | 2.3 | 4.0 | 1.0 | 0.2 | 0.0 | 1.0 | 0.1 |
| Vβ12 | 3.2 | 0.9 | 1.9 | 0.9 | 2.5 | 0.1 | 0.7 | 1.8 | 0.0 | 0.0 | 0.6 | 0.3 | 0.0 | 0.0 | 0.1 | 0.1 | 0.4 | 0.0 |
| Vβ13.1 | 2.4 | 0.3 | 4.6 | 10.4 | 1.5 | 0.5 | 2.6 | 4.2 | 0.0 | 0.1 | 0.8 | 0.0 | 8.1 | 10.0 | 5.6 | 16.5 | 5.0 | 0.0 |
| Vβ13.2 | 3.1 | 0.2 | 1.4 | 0.4 | 1.8 | 0.2 | 0.2 | 0.1 | 0.0 | 0.0 | 0.2 | 0.6 | 0.3 | 1.1 | 0.3 | 0.0 | 0.5 | 99.2 |
| Vβ13.6 | 2.8 | 9.1 | 3.6 | 0.5 | 1.2 | 0.1 | 0.2 | 0.1 | 0.2 | 0.1 | 0.2 | 0.0 | 1.7 | 0.1 | 3.2 | 6.9 | 0.4 | 0.0 |
| Vβ14 | 1.9 | 1.4 | 2.0 | 3.6 | 1.3 | 63.3 | 0.4 | 0.7 | 0.0 | 0.0 | 1.0 | 5.3 | 3.2 | 2.8 | 0.6 | 1.4 | 0.4 | 0.1 |
| Vβ16 | 2.4 | 1.0 | 6.9 | 6.9 | 3.3 | 0.3 | 4.1 | 1.1 | 0.0 | 0.1 | 0.8 | 0.0 | 0.1 | 0.3 | 0.1 | 25.4 | 0.5 | 0.0 |
| Vβ17 | 4.6 | 0.1 | 2.9 | 1.8 | 0.8 | 0.0 | 0.2 | 0.0 | 0.0 | 0.0 | 1.5 | 0.0 | 1.0 | 32.9 | 1.3 | 0.1 | 12.4 | 0.0 |
| Vβ18 | 2.4 | 0.1 | 0.2 | 0.0 | 0.5 | 0.0 | 0.1 | 0.0 | 0.4 | 0.0 | 0.4 | 0.2 | 1.0 | 0.2 | 3.6 | 0.0 | 0.4 | 0.0 |
| Vβ20 | 7.0 | 5.9 | 6.6 | 13.8 | 4.6 | 0.1 | 3.7 | 0.1 | 0.0 | 0.4 | 0.7 | 0.2 | 0.1 | 0.0 | 1.4 | 0.4 | 0.1 | 0.0 |
| Vβ21.3 | 0.4 | 0.5 | 3.8 | 4.3 | 0.4 | 0.1 | 0.1 | 0.1 | 0.0 | 3.2 | 6.2 | 0.1 | 2.4 | 0.0 | 0.6 | 0.3 | 0.5 | 0.0 |
| Vβ22 | 11.1 | 0.2 | 3.3 | 1.7 | 2.8 | 0.2 | 6.2 | 1.8 | 0.1 | 0.0 | 15.1 | 2.1 | 0.2 | 2.6 | 1.4 | 0.6 | 1.9 | 0.7 |
| Vβ23 | 3.7 | 0.3 | 0.4 | 0.1 | 0.8 | 0.8 | 0.9 | 0.5 | 1.6 | 0.5 | 0.1 | 4.6 | 0.1 | 0.1 | 0.2 | 6.7 | 0.1 | 0.0 |

| | Panc 10 | | Panc 11 | | Panc 12 | | Panc 13 | | Panc 14 | | Panc 15 | | Panc 16 | | Panc 17 | |
|---|---|---|---|---|---|---|---|---|---|---|---|---|---|---|---|---|
| | CD4 | CD8 | CD4 | CD8 | CD4 | CD8 | CD4 | CD8 | CD4 | CD8 | CD4 | CD8 | CD4 | CD8 | CD4 | CD8 |
| Vβ1 | 4.4 | 7.1 | 13.0 | 0.5 | 1.4 | 25.8 | 0.1 | 0.3 | 0.9 | 16.2 | 0.6 | 2.8 | 0.3 | 0.4 | 2.2 | 5.7 |
| Vβ2 | 4.1 | 39.8 | 2.4 | 0.2 | 7.5 | 4.8 | 1.5 | 0.6 | 5.1 | 0.7 | 29.3 | 1.5 | 48.9 | 1.0 | 16.3 | 1.1 |
| Vβ3 | 3.7 | 1.3 | 0.0 | 0.0 | 1.7 | 8.8 | 0.0 | 0.0 | 13.3 | 2.0 | 1.1 | 17.9 | 11.7 | 0.2 | 17.8 | 2.6 |
| Vβ4 | 6.0 | 0.1 | 0.0 | 0.0 | 2.2 | 0.4 | 4.8 | 0.0 | 0.9 | 0.1 | 8.3 | 1.2 | 0.1 | 0.1 | 0.0 | 0.0 |
| Vβ5.1 | 10.8 | 1.1 | 0.6 | 0.7 | 3.4 | 0.5 | 4.6 | 0.2 | 2.7 | 0.8 | 4.2 | 1.6 | 15.2 | 0.2 | 7.7 | 0.0 |
| Vβ5.2 | 1.3 | 9.4 | 0.1 | 0.1 | 0.3 | 1.8 | 0.1 | 0.1 | 0.4 | 0.1 | 2.3 | 0.8 | 1.2 | 0.1 | 0.4 | 0.0 |
| Vβ5.3 | 0.7 | 18.0 | 0.2 | 0.0 | 0.3 | 1.6 | 0.1 | 0.0 | 0.1 | 0.3 | 4.2 | 0.3 | 0.9 | 0.0 | 1.4 | 0.4 |
| Vβ7.1 | 0.5 | 1.5 | 0.0 | 0.0 | 1.6 | 2.8 | 0.1 | 0.3 | 0.3 | 6.3 | 0.9 | 1.8 | 0.3 | 2.2 | 1.1 | 1.5 |
| Vβ7.2 | 1.6 | 0.1 | 0.0 | 0.0 | 0.4 | 0.1 | 0.0 | 0.0 | 0.7 | 4.8 | 0.4 | 0.1 | 0.0 | 0.0 | 2.2 | 1.4 |
| Vβ8 | 2.7 | 1.4 | 0.4 | 0.3 | 3.6 | 1.8 | 1.1 | 0.5 | 1.7 | 0.8 | 1.9 | 4.1 | 0.5 | 0.3 | 2.7 | 1.9 |
| Vβ9 | 3.4 | 1.2 | 0.5 | 0.2 | 6.7 | 1.0 | 2.4 | 0.1 | 9.7 | 2.3 | 2.0 | 2.0 | 1.8 | 0.4 | 1.9 | 1.5 |
| Vβ11 | 0.6 | 0.3 | 0.1 | 0.2 | 0.8 | 0.3 | 0.2 | 0.2 | 0.3 | 0.2 | 1.4 | 0.7 | 0.4 | 0.2 | 0.2 | 0.0 |
| Vβ12 | 1.6 | 0.5 | 0.0 | 0.0 | 0.3 | 1.0 | 0.3 | 0.0 | 1.5 | 5.9 | 0.3 | 0.2 | 0.3 | 0.1 | 4.8 | 0.7 |
| Vβ13.1 | 3.7 | 0.1 | 35.5 | 0.1 | 9.2 | 0.3 | 0.5 | 0.2 | 4.3 | 19.6 | 0.7 | 0.8 | 2.6 | 1.1 | 1.5 | 0.9 |
| Vβ13.2 | 6.2 | 0.7 | 0.2 | 0.0 | 2.5 | 1.4 | 0.2 | 0.2 | 4.4 | 0.9 | 1.8 | 23.0 | 0.4 | 0.6 | 3.2 | 3.4 |
| Vβ13.6 | 2.0 | 0.3 | 0.6 | 0.0 | 1.5 | 0.2 | 0.5 | 0.2 | 0.3 | 1.9 | 0.6 | 0.9 | 0.3 | 0.4 | 1.1 | 2.0 |
| Vβ14 | 3.6 | 0.8 | 0.1 | 0.0 | 0.7 | 3.5 | 0.1 | 0.3 | 2.9 | 2.5 | 0.9 | 7.2 | 2.1 | 0.6 | 1.6 | 7.6 |
| Vβ16 | 1.0 | 0.2 | 0.0 | 0.2 | 0.1 | 0.1 | 0.0 | 0.2 | 0.2 | 0.8 | 0.2 | 0.2 | 0.1 | 0.0 | 1.3 | 0.0 |
| Vβ17 | 7.5 | 0.3 | 0.1 | 12.3 | 3.3 | 2.3 | 31.1 | 0.9 | 9.9 | 1.4 | 1.3 | 1.9 | 1.5 | 0.5 | 0.3 | 0.1 |
| Vβ18 | 0.5 | 0.2 | 0.1 | 0.2 | 0.3 | 0.3 | 0.5 | 0.1 | 0.9 | 3.1 | 3.3 | 3.6 | 0.4 | 3.3 | 0.3 | 0.0 |
| Vβ20 | 2.5 | 0.5 | 0.1 | 0.1 | 1.4 | 0.2 | 0.0 | 0.0 | 0.7 | 0.3 | 0.5 | 0.6 | 0.0 | 0.8 | 1.3 | 4.0 |
| Vβ21.3 | 2.4 | 0.1 | 0.9 | 0.0 | 0.5 | 8.0 | 0.5 | 0.0 | 1.2 | 0.5 | 0.4 | 0.4 | 0.2 | 1.6 | 1.5 | 6.8 |
| Vβ22 | 3.4 | 1.0 | 3.6 | 0.1 | 5.2 | 2.0 | 26.0 | 65.9 | 3.6 | 0.8 | 0.7 | 2.9 | 6.2 | 6.4 | 2.6 | 0.2 |
| Vβ23 | 4.5 | 0.7 | 0.6 | 0.1 | 0.2 | 0.4 | 0.3 | 1.0 | 0.5 | 0.7 | 1.8 | 7.4 | 0.3 | 0.3 | 1.1 | 2.0 |

TABLE 6
Vβ analysis and clonality in TILs after expansion

| | Cell | | Vβ by Flow | Number |
|---|---|---|---|---|
| Patient | population | Family by pcr | cytometry | of clones |
| Panc 1 | CD8 | TRBV9 | Vβ1 | 2 |
| Panc 5 | CD4 | TRBV12 | Vβ8 | 1 |
| Panc 7 | CD4 | TRBV5,1 | Vβ5,1 | 1 |
| | CD8 | TRBV19 | Vβ17 | 2 |
| | | TRBV28 | Vβ3 | 2 |
| Panc 8 | CD8 | TRBV14 | Vβ16 | 1 |
| Panc 9 | CD8 | TRBV6 | Vβ13,2 | 1 |
| Panc 10 | CD8 | TRBV20,1 | Vβ2 | 2 |
| Panc 11 | CD8 | TRBV20 | Vβ2 | 1 |
| | | TRBV7,3/6/7/9 | No antibody | 1 |
| | | TRBV19 | Vβ17 | 1 |
| Panc 13 | CD4 | TRBV19 | Vβ17 | 1 |
| Panc 14 | CD8 | TRBV9 | Vβ1 | 2 |
| Panc 15 | CD4 | TRBV20,1 | Vβ2 | 2 |
| | CD8 | TRBV6,2 | Vβ13,2 | 2 |
| Panc 16 | CD4 | TRBV20,1 | Vβ2 | 1 |
| | | TRBV7,3 | No antibody | 2 |
| | CD8 | TRBV7,2,8 | No antibody | 2 |
| | | TRBV19 | Vβ17 | 1 |

TABLE 7
CDR3 sequence analysis in TIL

| TIL | Cell | | | | | | Flow |
|---|---|---|---|---|---|---|---|
| ID | subset | Vβ | Vβ | (-D-) | Jβ | Jβ | AB |
| Panc | CD8 | TRBV9 | CAS | SVQG | ETQYF | TRBJ2- | Vβ1 |
| 1 | | | | VSQ | | 5 | |
| Panc | CD4 | TRBV12- | CAS | SRRG | DTQYF | TRBJ2- | Vβ8 |
| 5 | | 3/4 | | SG | | 3 | |
| Panc | CD4 | TRBV5.1 | CAS | STDSN | TEAFF | TRBJ | Vβ5.1 |
| 7 | | | | | | 1-1 | |
| | CD8 | TRBV28 | CAS | SFQT | DTQYF | TRBJ2- | Vβ3 |
| | | | | AHT | | 3 | |
| Panc | CD8 | TRBV14 | CAS | SLRDS | DEAFF | TRBJ | Vβ16 |
| 8 | | | | | | 1-1 | |
| Panc | CD8 | TRV6- | CAS | SLQ | DEQFF | TRBJ2- | NA |
| 9 | | 2/3 | | GRV | | 1 | |
| Panc | CD4 | TRBV20- | CSA | RDG | TEAFF | TRBJ1- | Vβ2 |
| 16 | | 1 | | TLN | | 1 | |
| | | TRBV20- | CSA | RPLR | HGYTF | TRBJ1- | Vβ1 |
| | | 1 | | DRVA | | 2 | |
| | CD8 | TRBV19- | CAS | SILA | ETQYF | TRBJ2- | Vβ17 |
| | | 1 | | FRAG | | 5 | |
| | | TRBV7- | CAS | SQG | YEQYF | TRBJ2- | NA |
| | | 6 | | PN | | 7 | |
| Panc | CD8 | TRBV7-2 | CAS | SFGL | NEQFF | TRBJ2- | NA |
| 17 | | | | AGA | | 1 | |
| | | TRBV7-2 | CAS | SSRL | NEQFF | TRBJ2- | NA |
| | | | | AGTY | | 1 | |

Example 7—Recognition of known TAAs by the Individual T-Cell Products from Pancreas Cancer Patients

TIL were exposed to commonly shared antigens for three days, in the presence of the blocking MHC class I (W6/32) or HLA-DR (L243) mAb, followed by IFNγ production analysis in the supernatant. Strong reactivity to Mesothelin that could be blocked with mAbs. Note that Panc 10 is predominantly a CD4+ TIL that could be blocked with the anti-HLA-DR mAb and not with the MHC class I—directed reagent.

We have also included an isotype control (anti-C1q, 242G3 mAb) that showed similar results as the antigen stimulation without the blocking mAb. Results are means of triplicates. Increased reactivity upon L243 (e.g. Panc16) may reflect T-cell stimulation of crosslinking HLA-DR molecules on activated T-cells. Only positive results are reported.

TABLE 8
TAA IFNγ production (pg/mL) in TIL in response to commonly shared TAAs

| | | NY- | NY- | | | | | | |
|---|---|---|---|---|---|---|---|---|---|
| | NY- | ESO1 + | ESO1 + | | Survivin + | Survivin + | | Mesothelin + | Mesothelin + |
| | ESO1 | W6/32 | L243 | Survivin | W6/32 | L243 | Mesothelin | W6/32 | L243 |
| Panc 1 | 6.97 | 0 | 0 | 0 | 0 | 0 | 262.23 | 15.26 | 0 |
| Panc 2 | 24.20 | 0 | 0 | 0 | 0 | 0 | 291.27 | 13.50 | 0 |
| Panc 10 | 4.13 | 0 | 0 | 66.06 | 0 | 0 | 78.70 | 72.88 | 0 |
| Panc 11 | 0 | 0 | 0 | 0 | 0 | 0 | 131.16 | 0 | 0 |
| Panc 14 | 10.47 | 0 | 0 | 0 | 0 | 0 | 275.15 | nd | 249.95 |
| Panc 16 | 173.19 | 0 | 0 | 8.35 | 0 | 0 | 19.64 | 0 | 60.84 |

### REFERENCES

1. Rosenberg S A, Restifo N P, Yang J C, Morgan R A, Dudley M E. Adoptive cell transfer: a clinical path to effective cancer immunotherapy. Nature reviews Cancer. 2008; 8(4):299-308.

2. Rosenberg S A, Packard B S, Aebersold P M, Solomon D, Topalian S L, Toy S T, et al. Use of tumor-infiltrating lymphocytes and interleukin-2 in the immunotherapy of patients with metastatic melanoma. A preliminary report. The New England journal of medicine. 1988; 319(25):1676-80.

3. Schwartzentruber D J, Hom S S, Dadmarz R, White D E, Yannelli J R, Steinberg S M, et al. In vitro predictors of therapeutic response in melanoma patients receiving tumor-infiltrating lymphocytes and interleukin-2. Journal of clinical oncology : official journal of the American Society of Clinical Oncology. 1994; 12(7):1475-83.

4. Pittet M J, Speiser D E, Valmori D, Cerottini J C, Romero P. Cutting edge: cytolytic effector function in human circulating CD8+ T cells closely correlates with CD56 surface expression. Journal of immunology. 2000; 164(3):1148-52.

5. ShenX, Zhou J, Hathcock K S, Robbins P, Powell D J, Jr., Rosenberg S A, et al.Persistence of tumor infiltrating lymphocytes in adoptive immunotherapy correlates with telomere length. Journal of immunotherapy. 2007; 30(1):123-9.

6. Zhou J, Shen X, Huang J, Hodes R J, Rosenberg S A, Robbins P F. Telomere length of transferred lymphocytes correlates with in vivo persistence and tumor regression in melanoma patients receiving cell transfer therapy. Journal of immunology. 2005; 175(10):7046-52.

7. Green M Sambrook J, Molecular Cloning: A Laboratory Manual, Fourth Edition. Cold Spring Harbor Press. 2012.

8. Wingfield, P T. Production of Recombinant Proteins. Current Protocols in Protein Science. 2010.

9. Robbins P F, Morgan R A, Feldman S A, Yang J C, Sherry R M, Dudley M E, Wunderlich J R, Nahvi A V, Heiman L J, Mackall C L, Kammula U S, Hughes M S, Restifo N P, Raffeld M, Lee C C, Levy C L, Li Y F, EI-Gamil M, Schwarz S L, Laurencot C, Rosenberg S A. Tumor regression in patients with metastatic synovial cell sarcoma and melanoma using genetically engineered lymphocytes reactive with NY-ESO-1. Journal of Clinical Oncology. 2011; 29(7):917-924.

10. Kershaw M, Westwood JA,. Darcy PK. Gene-engineered T cells for cancer therapy. Nature Reviews Cancer, 2013; 13:525-541.

11. Johnson L A, Heemskerk B, Powell D J Jr, Cohen C J, Morgan R A, Dudley M E, Robbins PF, Rosenberg S A. Gene transfer of tumor-reactive TCR confers both high avidity and tumor reactivity to nonreactive peripheral blood mononuclear cells and tumor-infiltrating lymphocytes. Journal of Immunology. 2006; 177(9):6548-6559.

12. Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mavroukakis S A, Rosenberg S A. Cancer regression in patients after transfer of genetically engineered lymphocytes. Science. 2006; 314(5796):126-129.

1. A protein comprising a Vbeta sequence with an amino acid sequence with a sequence identity of at least 90% to a sequence selected from SEQ ID NO: 1 to 26.

2. The protein according to claim 1, wherein the sequence identity is at least 95%.

3. The protein according to claim 1, wherein the protein is a recombinant protein.

4. The protein according to claim 1, wherein the protein is a T-cell receptor (TCR).

5. A T-cell comprising a TCR according to claim 4.

6. A method of treating cancer in a subject comprising administering a T cell according to claim 5 to a subject having cancer.

7. A method of selecting a T-cell product for use in active immunotherapy in a patient, wherein the method comprises the following steps:
a) providing one or more T-cell products;
b) identifying the presence of a TCR comprising a sequence with an identity of at least 95% to the sequence selected from SEQ ID NO: 1 to 27 in the one or more T-cell products; and
c) selecting the T-cell product comprising a TCR identified in step b) for use in therapy.

8. The method according to claim 7, wherein the T-cell product is a T-cell population.

9. The method according to claim 7, further comprising determining the clonality of the Vβ family to which the TCR belongs and selecting the T-cell product in which the TCR is present and the Vβ family to which the TCR belongs is monoclonal.

10. The method according to claim 7, further comprising determining the percentage of the TCR and/or the Vβ family to which the TCR belongs in the T-cell product and selecting the T-cell product in which the percentage of the TCR is the highest.

11. The method according to claim 7, wherein the patient suffers from cancer.

12. The method according to claim 11, wherein the cancer is selected from glioblastoma and pancreas cancer.

13. The method according to claim 7, wherein providing the one or more T-cell products comprises:
providing a body sample containing T-cells from the patient;
culturing cells of the sample in-vitro in the presence of at least one cytokine, and optionally an expansion antigen to expand the T-cells.

14. The protein according to claim 1, wherein the sequence identity is at least 98%.

15. The protein according to claim 1, wherein the sequence identity is 100%.

16. The method according to claim 13, wherein the at least one cytokine is interleukin 2 (IL-2), interleukin 15 (IL-15) and interleukin 21 (IL-21).

17. The method of claim 6, wherein the cancer is glioblastoma or pancreatic cancer.


SEQUENCE LISTING (26 sequences)

SEQ ID NO: 1 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSAATGDRPYEQYFGPGTRLTVT

SEQ ID NO: 2 - protein, 112 aa, Homo sapiens
DAGITQSPRHKVTETGTPVTLRCHQTENHRYMYWYRQDPGHGLRLIHYSYGVKDTDKGEVSDGYSVSRSKTEDFLLTLESATSSQTSVYFCAIRTGSDNEQSFGPGTRLTVL

SEQ ID NO: 3 - protein, 113 aa, Homo sapiens
EAEVAQSPRYKITEKSQAVAFWCDPISGHATLYWYRQILGQGPELLVQFQDESVVDDSQLPKDRFSAERLKGVDSTLKIQPAELGDSAMYLCASRYTGSIEQFFGPGTRLTVL

SEQ ID NO: 4 - protein, 115 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSAGTSGVTYEQYFGPGTRLTVT

SEQ ID NO: 5 - protein, 110 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSTRFEQYFGPGTRLTVT

SEQ ID NO: 6 - protein, 110 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSTRFEQYFGPGTRLTVT

SEQ ID NO: 7 - protein, 110 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSTRFEQYFGPGTRLTVT

SEQ ID NO: 8 - protein, 116 aa, Homo sapiens
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSSRRDHTYNGQFFGPGTRLTVL

SEQ ID NO: 9 - protein, 115 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSRRDHTYNGQFGPGTRLTVL

SEQ ID NO: 10 - protein, 113 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLQGANYGYTFGSGTRLTVV

SEQ ID NO: 11 - protein, 121 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARVIPSGGVVQGTDTQYFGPGTRLTV

SEQ ID NO: 12 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSWDKSYEQYFGPGTRLTVT

SEQ ID NO: 13 - protein, 117 aa, Homo sapiens
EAEVAQSPRYKITEKSQAVAFWCDPISGHATLYWYRQILGQGPELLVQFQDESVVDDSQLPKDRFSAERLKGVDSTLKIQPAELGDSAMYLCASSLSRLALFSYEQYFGPGTRLTVT

SEQ ID NO: 14 - protein, 114 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVQGVSQETQYFGPGTRLLVL

SEQ ID NO: 15 - protein, 114 aa, Homo sapiens
DAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSRRGSGDTQYFGPGTRLTVL

SEQ ID NO: 16 - protein, 114 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRRGSGDTQYFGPGTRLTVL

SEQ ID NO: 17 - protein, 112 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSTDSNTEAFFGQGTRLTVV

SEQ ID NO: 18 - protein, 114 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASSFQTAHTDTQYFGPGTRLTVL

SEQ ID NO: 19 - protein, 112 aa, Homo sapiens
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSLRDSDEAFFGQGTRLTV

SEQ ID NO: 20 - protein, 113 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSLQGRVDEQFFGPGTRLTVL

SEQ ID NO: 21 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDGTLNTEAFFGQGTRLTVV

SEQ ID NO: 22 - protein, 118 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARPLRDRVAHGYTFGSGTRLTVV

SEQ ID NO: 23 - protein, 115 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSILAFRAGETQYFGPGTRLLVL

SEQ ID NO: 24 - protein, 113 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSQGPNYEQYFGPGTRLTVT

SEQ ID NO: 25 - protein, 115 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFGLAGANEQFFGPGTRLTVL

SEQ ID NO: 26 - protein, 116 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSSRLAGTYNEQFFGPGTRLTVL